SEQUENCE LISTING (2 sequences)

SEQ ID NO: 1 - DNA, 3900 bp, Candida bombicola
atggtggatgatatacaggtagagaagcgtgagaaactcatcgagactaaggacaagcttctcgaggagaagctctctgcgttagatccacatgaggccaatgtattgcgaagtcagcttgaaacaaagagagtcgccacaagctttttcaggttgttcagattttgcactccccttgacgttttcttggagatacttgcgcttttttttgcagcggtgcatggagccgcgcttccaatgttcacgttagtagtgggcgccatcttcaacacattcagagacttcacttcatatgacctcaagggcaatgagttccagcataaggtgaatcacctgtctctctattttgtctatattggcattggtatgctcggcagtgcgtttctcgagagcttcctgcttgtggacagaggcgaagtgttggcaggacgttaccgaaagcattatctgagtgctgttattcgccagaatatcgcgttttacgacaaactaggtggtggcgaggtcagcaccagaatcattaacgataccaactcaattcaggaagcgatcagcgacaagcttggaaacgtcgtacagggaatagcttccttcattgcggccaccgttataagttttgcttcgcaatggaaactggcttgcatcctcctgagtgctgtagggttcatggtaatcacaatgggaactggcgccaccttcatggccaaatatcagctcagatctgacgcgatatattcgcagtctggagctaccgttgcggaggaggctctcagtgctgtcaggactacagtagcatttggcgctcaacctcatctcgccgtcaagtatgaaaaggtacttgatcgtgttgtgaaggaatcgaagcggagcagttactcattgggggtcatgttagcgtgcatatgggctagtactttttgggtgtatgccttagctctgtggcagggttccagagaaatcgttagtgggagtgctgacgttggaaagataatagttgtaatcacagctatgttacttggaagcttccagcttgggaatatcgcgccaaacgtgaggtttcttgtcaagggtctcactgccgcgagcattctcaatgaggccattgatcgtgtcccagtcatcgatggccagtccatagataaaggaattgtcccccaaactaaggccgttggcagaattgagctcaaaaatgtcaagttccgatatcctagtcgcccagacgttttggtcctctccgattttagccttgaagttcctgctggatctactgtggcactggtaggtgcctcgggatcagggaagtctacaattgtaggtattcttgagaggttctatttacctctcgaaggaagcgttactctggatggccaggagattagcgacctgaacacaagatggctccgtcaacaaattggttatgttcagcaggaaccagtactcttttcagagtcaatatatgagaatatcagctatggtttgattggcactgacattgagttcgctgacgagcatgttaaggaagctaaaatcattcaagcttgtaaagatgccaatgcctgggatttcattcagactctctcagaaggcatccaaaccaatgttggagatcgaggatttcttctcagcggtggtcagaaacaacgcattgcaatagcaagagcaatcgtctcagaccctaaaattctgctgctcgatgaagcgacttctgctctggataccaaatctgaaggtatcgttcaagatgcgctcgacaaagcggccgaaggtcgtaccactatagtcgttgcacacagactctctacgatcaaggatgccaacaagatagttgtcatgtctaaaggtaacgtcatagagcagggtactcacaatgagctcatacagcgagaagggccttataaagctttggttgatgctcaaagagtaactaaagcaaagagcactaacgttgaggtcctcgatattgaagctctagacatttcgcctctggactcactgaacgaaaagttcaatcccaaggatgtgagcacattgagtgttcacagtgcaggtactcagaccactcaacctcctgaatatcaagaaaatgacatccctggtgtgcgcaaccccccacatagcacgttgatgaccaataccaaactggtttgggggctgaataggaaagaatggggttacattctcattggtagtttagcctccattattttgggctattgctatcctgcaatggcaataataactggccaaaccactggaagcatggttctacctcccagtgaatacggaaaaatgcggcatgtggtgaatatcatgggatggtggtattttttcgtaggctgcatttcattcatgacggcttttatcactatagctgctttatcacttgcatctgataagttggtcaaaaatatcagattagctttgttccgccaattgatgcgaatggatattgcattcttcgaccacaaaaacaacacgccgggtgcgctaacctcaattttggcgaaggaagctaaaatgatcgagggtttgagtggggccaccctcggtcaaattcaacagagtctggtgaccttgattggcggcatagttactggtatacctttcaattggagaattggactcgtggctacgtctgttgttcctgtcatgttggtgtgtggcttcgtcagagtctgggttcttacccaattatcggatcgtgcgagagaagtttacgaacgaagtggctccatggcatctgagtatacaagtgctgtccgcacagtccagtccttaactcgtgagttagacgtggtcgtaaaatacacaaagacagtagactctcagattttcagctccagaattgccattgcccgctcagcattgtactacgcactctcggaaggaatgacaccctgggtggtagccctcgttttttggtggggaagcactgtaatgagacgaggtgaagcttcggtcgcaggatatatgactgtcttcatggctattattacaggttctcaagccgctggccaaattttcagctatgctccaaacatgaactcagccaaagatgcagcgcgtaacatttacagaatcttgactgccactccttctatagatgtatggagtgaggaaggttacgttgctcccgaggagtcggtgagaggagatattgagttccgtcatgtgaatttccgatatcctactcgacctcaagtaccagttttacaagatctcaacttaacagtcaaaaagggccaatacatcgctctagttggagccagtggatgcggtaagtctactactattggactggtggaaagattttatgatccattagcaggtcaagtacttttcgatgggaaagatttacgcgaatataacctgaatgcattgagatcacacattgctttagtccagcaagaaccaatgctttattcaggcacgctacgtgagaatattctaatgggatggtctggccctgagtctgaagtaacgcaggagatgattgaggatgccgctcgcaaagcgaacattcacgaattcatcatgtcgttgcctgatggctacgaaacgctcagcggatctaggggatcgttgctatctggggggcaaaagcagcgaattgcaattgcaagggccctgatcagaaatccaaaggtactcctcctcgatgaggccacctcagctctggattccgaatctgagaaagtagttcaagcagcactcgacgcagcagcgaagggccgtactacaatcgccgttgcgcatagattatcaacaattcagaaagcagatgtcatatatgtgttctcaggagggcgcatcgtggagcagggcgaccatcagagcctccttgaactcaatggatggtacgctgaattggtgaacttgcaaggtctcggagagatttga

SEQ ID NO: 2 - protein, 1299 aa, Candida bombicola
  MISC_FEATURE (352)..(648): NBD1
MVDDIQVEKREKLIETKDKLLEEKLSALDPHEANVLRSQLETKRVATSFFRLFRFCTPLDVFLEILALFFAAVHGAALPMFTLVVGAIFNTFRDFTSYDLKGNEFQHKVNHLSLYFVYIGIGMLGSAFLESFLLVDRGEVLAGRYRKHYLSAVIRQNIAFYDKLGGGEVSTRIINDTNSIQEAISDKLGNVVQGIASFIAATVISFASQWKLACILLSAVGFMVITMGTGATFMAKYQLRSDAIYSQSGATVAEEALSAVRTTVAFGAQPHLAVKYEKVLDRVVKESKRSSYSLGVMLACIWASTFWVYALALWQGSREIVSGSADVGKIIVVITAMLLGSFQLGNIAPNVRFLVKGLTAASILNEAIDRVPVIDGQSIDKGIVPQTKAVGRIELKNVKFRYPSRPDVLVLSDFSLEVPAGSTVALVGASGSGKSTIVGILERFYLPLEGSVTLDGQEISDLNTRWLRQQIGYVQQEPVLFSESIYENISYGLIGTDIEFADEHVKEAKIIQACKDANAWDFIQTLSEGIQTNVGDRGFLLSGGQKQRIAIARAIVSDPKILLLDEATSALDTKSEGIVQDALDKAAEGRTTIVVAHRLSTIKDANKIVVMSKGNVIEQGTHNELIQREGPYKALVDAQRVTKAKSTNVEVLDIEALDISPLDSLNEKFNPKDVSTLSVHSAGTQTTQPPEYQENDIPGVRNPPHSTLMTNTKLVWGLNRKEWGYILIGSLASIILGYCYPAMAIITGQTTGSMVLPPSEYGKMRHVVNIMGWWYFFVGCISFMTAFITIAALSLASDKLVKNIRLALFRQLMRMDIAFFDHKNNTPGALTSILAKEAKMIEGLSGATLGQIQQSLVTLIGGIVTGIPFNWRIGLVATSVVPVMLVCGFVRVWVLTQLSDRAREVYERSGSMASEYTSAVRTVQSLTRELDVVVKYTKTVDSQIFSSRIAIARSALYYALSEGMTPWVVALVFWWGSTVMRRGEASVAGYMTVFMAIITGSQAAGQIFSYAPNMNSAKDAARNIYRILTATPSIDVWSEEGYVAPEESVRGDIEFRHVNFRYPTRPQVPVLQDLNLTVKKGQYIALVGASGCGKSTTIGLVERFYDPLAGQVLFDGKDLREYNLNALRSHIALVQQEPMLYSGTLRENILMGWSGPESEVTQEMIEDAARKANIHEFIMSLPDGYETLSGSRGSLLSGGQKQRIAIARALIRNPKVLLLDEATSALDSESEKVVQAALDAAAKGRTTIAVAHRLSTIQKADVIYVFSGGRIVEQGDHQSLLELNGWYAELVNLQGLGEI

(19) United States
(12) Patent Application Publication
(10) Pub. No.: US 2012/0311741 A1
(43) Pub. Date: Dec. 6, 2012

(54) SOPHOROLIPID TRANSPORTER PROTEIN

(76) Inventors: Wim Soetaert, Lovendegem (BE); Inge Van Bogaert, Laarne (BE)

(21) Appl. No.: 13/514,908

(22) PCT Filed: Dec. 9, 2010
(86) PCT No.: PCT/EP2010/069280

§ 371 Date: Aug. 20, 2012

(30) Foreign Application Priority Data
Dec. 11, 2009 (GB) 0921691.2

(51) Int. Cl. C12N 15/80; C12N 15/81; C12N 1/19; A01H 15/00; C07K 14/40; C12N 15/31

(52) U.S. Cl. 800/281; 530/350; 536/23.74; 435/254.22; 800/297; 435/471

(57) ABSTRACT

The invention relates to a transporter protein involved in the transport of sophorolipids. More specifically, it relates to a Candida bombicola sophorolipid transporter protein, and the use of this transporter to modulate the secretion and/or production of glycolipids, preferably sophorolipids in organisms, preferably in fungi.

### CROSS-REFERENCE TO RELATED APPLICATIONS

This is a national phase entry under 35 U.S.C. §371 of International Patent Application PCT/EP2010/06980, filed Dec. 9, 2010, published in English as International Patent Publication WO 2011/070113 A1 on Jun. 16, 2011, which claims the benefit under Article 8 of the Patent Cooperation Treaty to Great Britain Patent Application Serial No. 0921691.2, filed Dec. 11, 2009.

### TECHNICAL FIELD

The invention relates to a transporter protein involved in the transport of sophorolipids. More specifically, it relates to a Candida bombicola sophorolipid transporter protein, and the use of this transporter to modulate the secretion and/or production of glycolipids, preferably sophorolipids in organisms, preferably in fungi.

### BACKGROUND

Candida bombicola (Torulopsis bombicola, teleomorph: Starmerella bombicola) is a non-pathogenic yeast that shows the unusual capacity to produce biosurfactants, more precisely sophorolipids, at very high and economic relevant titers. Those sophorolipids show a broad application range; they can be used as a detergent or emulsifier in various industries where they offer a bio-based and environmentally friendly alternative for the chemical-derived surfactants (e.g., in cleaning applications, cosmetic formulations, paints, etc.). Furthermore, they show biological activity: they possess antimicrobial and immune-stimulating properties and even display anti-HIV and cell-differentiating activities (reviewed by Van Bogaert et al., 2007).

Despite the potential industrial importance of this strain and its sophorolipids, very little is known about the biochemical synthesis, its regulation and related pathways. For instance, it is not clear how the sophorolipids are excreted in such high amounts (up to 400 g/L) into the culture medium; vesicles could be involved, but the process might as well be mediated by active or passive transporters. However, up to now, there was no indication that such transporter existed.

### SUMMARY OF THE INVENTION

Recently, we identified a gene in the C. bombicola ATTC 22214 genome, the corresponding gene product of which showed some similarity (51% identity or lower, as measured by BLASTp) with ABC Multidrug Resistance transporters (MDR). For some of those MDR genes, experimental data about their function was available; all of these were involved in fungal antibiotic production and protection against cytotoxic agents (e.g., Andrade et al., 2000, Tobin et al., 1997). However, deletion of the C. bombicola gene does not affect the resistance of the host strain against antibiotics, indicating that the C. bombicola gene does not encode a MDR protein sensu stricto, and should have another function in the yeast. Surprisingly, we found that the corresponding Candida bombicola gene product is involved in sophorolipid excretion, and that the gene can be used to modulate sophorolipid production and/or excretion.

Described is an isolated sophorolipid transporter protein. Sophorolipids are known to the person skilled in the art and are described, amongst others, by Van Bogaert et al. (2007), hereby incorporated herein by reference. A sophorolipid transporter protein, as used here, is a membrane protein involved in the active or passive secretion of sophorolipids. The terms “protein” and “polypeptide” as used in this application are interchangeable. “Protein” refers to a polymer of amino acids and does not refer to a specific length of the molecule. This term also includes post-translational modifications of the polypeptide, such as glycosylation, phosphorylation and acetylation.

Preferably, the protein has at least 70% identities, preferably 75% identities, more preferably 80% identities, even more preferably 85% identities, even more preferably 90% identities, even more preferably 95% identities to the full length of SEQ ID NO:2, as measured by BLASTp (Altschul et al., 1997; Altschul et al., 2005). Most preferably, the protein has a protein sequence as depicted in SEQ ID NO:2. Preferably, the transporter protein is isolated from a fungal species, preferably Candida species, preferably from Candida bombicola.

Also described is a nucleic acid sequence encoding a sophorolipid transporter protein according to the invention, or a functional fragment thereof. “Nucleic acid sequence,” “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA, including the antisense RNA. It also includes known types of modifications, for example, methylation, “caps” substitution of one or more of the naturally occurring nucleotides with an analog. A “functional fragment” as used here is any fragment with biological activity. One preferred embodiment of a functional fragment is the coding sequence. “Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to, mRNA, cDNA, recombinant nucleotide sequences or genomic DNA, while introns may be present as well under certain circumstances.

Another preferred embodiment of a functional fragment is a RNAi, derived from the sequence, and useful for down-regulating the expression. Preferably, the nucleic acid sequence has at least 70% identities, preferably 75% identities, more preferably 80% identities, even more preferably 85% identities, even more preferably 90% identities, even more preferably 95% identities to the full length of SEQ ID NO:1 of the Sequence Listing, as measured by BLASTn (Zhang et al., 2000; Morgulis et al., 2008). Most preferably, the nucleic acid sequence according to the invention is the sequence depicted in SEQ ID NO:1, or a functional fragment thereof comprising at least the coding sequence.

Also described is a host organism transformed with a nucleic acid sequence hereof. The host organism can be any host organism, including but not limited to, mammalian cells, insect cells, bacterial cells, plant cells, fungal and yeast cells and algae. Preferably, the host organism is a fungal cell. Even more preferably, the fungal cell belongs to a genus selected from the group consisting of Candida, Starmerella, Wickerhamiella, Ustilago, Pseudozyma and Rhodotorula. Preferably, the cell is an Ustilago maydis or a Candida bombicola cell. Most preferably, the fungal cell is a Candida bombicola cell.

Still another aspect of the invention is the use of a sophorolipid transporter protein according to the invention, and/or a nucleic acid sequence according to the invention to modulate glycolipid secretion and/or production. Indeed, by influencing the secretion, the intracellular concentration of glycolipids will vary, influencing the production by feedback regulation. Preferably, the glycolipid is a sophorolipid or a cellobiose lipid, or a biochemical modification (e.g., altered acetylation pattern, modified or non-conventional fatty acid tail) thereof. Cellobiose lipids are, amongst others, described by Teichmann et al. (2007), hereby incorporated herein by reference. Even more preferably, the glycolipid is a sophorolipid. Preferably, the modulation is an increase in secretion and/or production. The modulation can, as a non-limiting example, be realized by knocking out the gene, or by overexpression of the gene encoding the sophorolipid transporter protein according to the invention.

Further described is a method for obtaining increased secretion and/or production of glycolipids in a host organism, comprising transformation of the host organism with a nucleic acid sequence according to the invention. Preferably, the glycolipid is a sophorolipid or a cellobiose lipid, or a biochemical modification (e.g., altered acetylation pattern, non-conventional fatty acid tail) thereof. Even more preferably, the glycolipid is a sophorolipid. Preferably, the nucleic acid comprises the coding sequence encoding a sophorolipid transporter protein, according to the invention, operably linked to a strong promoter, which is functional in the host organism. “Operably linked” refers to a juxtaposition wherein the components so described are in a relationship permitting them to function in their intended manner. A promoter sequence “operably linked” to a coding sequence is ligated in such a way that expression of the coding sequence is achieved under conditions compatible with the promoter sequence. “Promoter” as used herein refers to a functional DNA sequence unit that, when operably linked to a coding sequence and possibly placed in the appropriate inducing conditions, is sufficient to promote transcription of the coding sequence. The host organism can be any host organism, including but not limited to, mammalian cells, insect cells, bacterial cells, plant cells, fungal and yeast cells and algae. Preferably, the host organism is a fungal cell. Even more preferably, the fungal cell belongs to a genus selected from the group consisting of Candida, Starmerella, Wickerhamiella, Ustilago, Pseudozyma and Rhodotorula. Preferably, the cell is an Ustilago maydis or a Candida bombicola cell. Most preferably, the fungal cell is a Candida bombicola cell.

### BRIEF DESCRIPTION OF THE FIGURES

FIG. 1: Scheme of the cassette allowing homologous recombination at the transporter locus. The original transporter promoter is replaced by the homologous glyceraldehyde 3-phosphate dehydrogenase (GPD) promoter.

FIG. 2: Constructed plasmid for the expression of the transporter in Ustilago maydis.

FIG. 3: Up: structural arrangements of MDR transporters (figure from Cannon et al., 2009). Down: transmembrane helix prediction according to Kroch et al. (2001). The 9^{th} helix was wrongly omitted, but if this one is kept into account, the inside and outside loops show a better fit to the (TM₆-NBD)₂ structure.

FIG. 4: Alignment of the first and second NBD. Conserved regions are shaded black.

FIG. 5: Sophorolipid production of the transporter knock-out mutants (MDR12, MDR21, and MDR31) and the wild-type strain on rapeseed oil.

FIG. 6: Sophorolipid production on rapeseed oil during stationary phase of the transporter over-expression strain and the wild-type strain.

FIG. 7: SEQ ID NO:1 start and stop codons are marked. GenBank accession number HQ660581.

FIG. 8: SEQ ID NO:2.

### DETAILED DESCRIPTION

### EXAMPLES

### Materials and Methods to the Examples

### Strains and Culture Conditions

Candida bombicola ATCC 22214 was used as the parental strain. Candida bombicola PT36, an ura3 autotrophic mutant, was derived from this parental strain (unpublished results) and used to construct both the knock-out and over-expression strains. U. maydis DSM17146 (MB215emt1) a strain deficient in mannosylerythritol lipid (MEL) production, was used in the heterologous expression experiments.

When sophorolipid production was intended, the medium described by Lang et al. (2000) was used. 37.5 g/L rapeseed oil was added two days after inoculation. Yeast cultures were incubated at 30° C. and 200 rpm for a total time of 10 days. Cellobiose lipid production was conducted according to the method described by Spoecker et al. (1999).

Antibiotic resistance of the mutants was tested on yeast peptone dextrose (YPD) plates (1% yeast extract, 2% peptone, 2% glucose and 2% agar) containing 50 μg/ml pleomycin or 400 or 800 μg/mL G418, or 300 μg/mL zeocin at pH 6.5 or 7. The different yeast cultures were grown o/n and put at the same optical density before ten-fold dilutions from 10⁻¹ till 10⁻⁵ were made. The plates ware incubated at 30° C. during several days and growth was monitored daily.

Escherichia coli XL10-Gold cells were used in all cloning experiments and were grown in Luria-Bertani (LB) medium (1% trypton, 0.5% yeast extract and 0.5% sodium chloride) supplemented with 100 mg/L ampicillin. Liquid E. coli cultures were incubated at 37° C. and 200 rpm.

### DNA Isolation and Sequencing

Yeast genomic DNA was isolated with the GenElute™ Bacterial Genomic DNA Kit (Sigma). Preceding protoplast formation was performed by incubation at 30° C. for 90 minutes with zymolyase (Sigma). U. maydis gDNA was isolated according to the protocol of De Maeseneire et al. (2007).

Bacterial plasmid DNA was isolated with the QIAprep Spin Miniprep Kit (Qiagen). All DNA sequences were determined at LGC Genomics (Berlin, Germany).

### Transformation

C. bombicola cells were transformed with the lithium acetate method (Gietz & Schiestl, 1995), but 50 mM LiAc was used instead of 100. Transformants were selected on synthetic dextrose (SD) plates (0.67% yeast nitrogen base without amino acids (DIFCO) and 2% glucose). E. coli cells were transformed as described by Inoue et al. (1990). Protoplast transformation of Ustilago maydis was carried out as described by Brachmann et al. (2004).

### Creation of the Knock-Out Cassette

The coding region of 3900 by and 386 and 521 by upstream and downstream of the sophorolipid transporter gene were amplified with the primers MDRtotFor and MDRtotRev, yielding a fragment of 4789 bp, which was cloned into the pGEM-T® vector (Promega). The created vector was digested with BglII, cutting the coding sequence of the gene twice, in this way deleting 2498 by of the transporter coding region.

The Candida bombicola Ura3 autotrophic marker (Van Bogaert et al., 2008) was inserted by means of the In-Fusion™ 2.0 Dry-Down PCR Cloning Kit (Clontech). The primers uraInfMdrFor and uraInfMdrRev were designed according to the guidelines of the manual and used for integration of the ura3 cassette (2091 bp) into the sophorolipid transporter gene.

The primer pair MDRtotFor and MDRtotREV were used for the amplification of a 4356 by fragment containing the ura3 marker with approximately 1 kb of the sophorolipid transporter sequence on each site, required for homologue recombination at the transporter locus. This linear fragment was used to transform Candida bombicola PT36.

Creation of the Candida bombicola Over-Expressing Strain

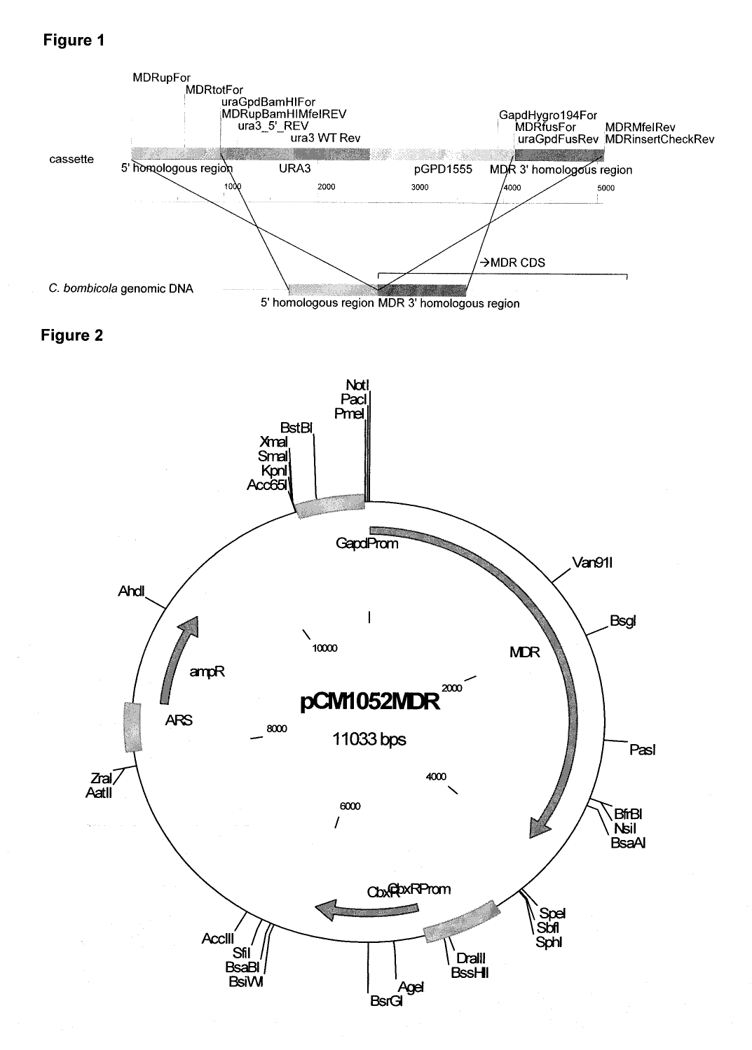

Over-expression of the MDR gene was achieved by replacing the original MDR promoter by the homologous glyceraldehyde 3-phosphate dehydrogenase (GPD) promoter. For this, a cassette allowing homologous recombination at the MDR locus was designed (FIG. 1). In a first step, the 5′ homologous region (969 bp) was amplified from C. bombicola gDNA with the primers MDRupFor and MDRupBamHIMfeIREV and cloned into the pGEM-T® vector (Promega). In a second step, the ura3 auxotrophic marker followed by the 1560 by GPD promoter region was amplified from pGEM-T_yEGFP_pGAPD1560 with the primers uraGpdBamHIFor and uraGpdFusRev. The resulting 3187 by fragment was linked by fusion PCR to the 3′ homologous region (974 bp), which was obtained by amplification with the primers MDRfusFor and MDRMfeIRev with C. bombicola gDNA as template. Both the vector obtained in the first step and the PCR fusion product achieved in the second step, were cut with BamHI-HF and MfeI (New England Biolabs) and a ligation was performed. The ligation mixture was transformed into competent E. coli cells and colonies were screened for the correct construct (total of 8078 bp) by colony PCR with the primers MDRtotFOR and ura3 wt REV. The plasmids of the colonies yielding a 1131 by fragment were isolated and sent for sequencing. The 5070 by integration cassette was amplified with the primers MDRupFor and MDRinsertChekREV and was used to transform C. bombicola PT36.

Creation of the Ustilago maydis Strain Expressing the Sophorolipid Transporter

The MDR coding sequence and its terminator of about 350 by was amplified from C. bombicola gDNA with the primers MDRctNotIFor and MDRctSpeIRev. The 4275 by fragment was cut with NotI and SpeI (New England Biolabs), as well as the vector pCM1052, which was kindly provided by Dr. William Holloman from the Cornell University Weill Medical College, New York, USA. A ligation was performed and the mixture was transformed into competent E. coli cells. Colonies were screened for the correct construct (total of 11033 bp; FIG. 2) by colony PCR with the primers MDRseq4 and hygroInsertCheckRev. U. maydis was transformed with either (1) the whole plasmid, (2) a 7073 by linear fragment derived by PCR with the primers MdrUmCasFor and HygroInsertCheckRev, or (3) a plasmid digested with KpnI and SfiI (New England Biolabs). Transformants were selected on YPD plates containing 40 μg/ml of carboxin (Sigma).

### Sampling

Analytical sophorolipid and cellobiose lipid samples were prepared as follows: 440 μL ethylacetate and 11 μL acetic acid were added to 1 mL culture broth and shaken vigorously for 5 minutes. After centrifugation at 9000 g for 5 minutes, the upper solvent layer was removed and put into a fresh Eppendorf tube with 600 μL ethanol. At the end of the incubation period, 3 volumes ethanol were added to the culture broth for total extraction of sophorolipids. Cell debris was removed by centrifugation at 1500 g during 10 minutes.

For further gravimetric analysis, the supernatant water-ethanol mixture was evaporated. Two volumes of ethanol were added to dissolve the sophorolipids and the residual hydrophobic carbon source. The mixture was filtrated to remove the water-soluble compounds and was evaporated again. One volume of water was added and set at pH 7, then 1 volume of hexane was added and, after vigorous shaking, the mixture was allowed to separate. The different fractions were collected, evaporated and the mass was determined. The hexane phase will contain residual oil, while the water phase contains the sophorolipids.

Samples were analyzed by HPLC and Evaporative Light Scattering Detection.

Cell dry weight (CDW) was measured by centrifugation of 2 mL culture broth for 5 minutes at 9000 g. Pellets were washed two times with ethanol to remove sophorolipids and hydrophobic substrate and finally dissolved in distilled water. The suspension was transferred to a cellulose nitrate filter with a pore diameter of 0.45 μm (Sartorius) and the dry weight was determined in the XM60 automatic oven from Precisa Instruments Ltd.

Glucose concentration in the culture supernatants was determined by analysis with the 2700 Select Biochemistry Analyzer (YSI Inc.).

Colony-forming units (CFU) were determined by plating decimal dilutions on agar plates with 10% glucose, 1% yeast extract and 0.1% urea, which were incubated at 30° C. for three days.

### HPLC Analysis of Glycolipids

Sophorolipid and cellobiose lipid samples were analyzed by HPLC on a Varian Prostar HPLC system using a Chromolith® Performance RP-18e 100-4.6 mm column from Merck KGaA at 30° C. and Evaporative Light Scattering Detection (Alltech). A gradient of two eluents, a 0.5% acetic acid aqueous solution and acetonitrile, had to be used to separate the components. The gradient started at 5% acetonitrile and linearly increased till 95% in 40 minutes. The mixture was kept this way for 10 minutes and was then brought back to 5% acetonitrile in 5 minutes. A flow rate of 1 mL/minute was applied. In order to be able to compare and quantify the different samples, dilutions of a standard were analyzed in parallel.

### Example 1

### Characterization of the Sophorolipid Transporter Sequence

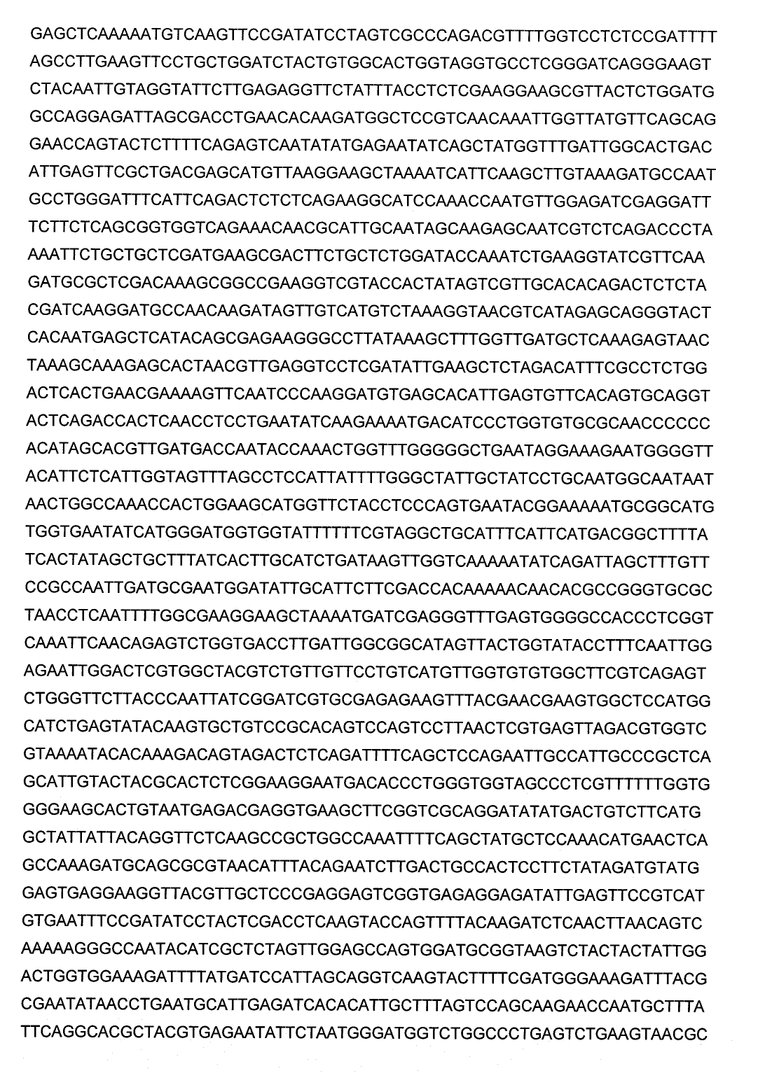

The sophorolipid transporter nucleotide sequence is given in FIG. 7 (SEQ ID NO:1). The sophorolipid transporter gene is found to be intron-less, just as most other C. bombicola genes (Van Bogaert et al., 2009a and b).

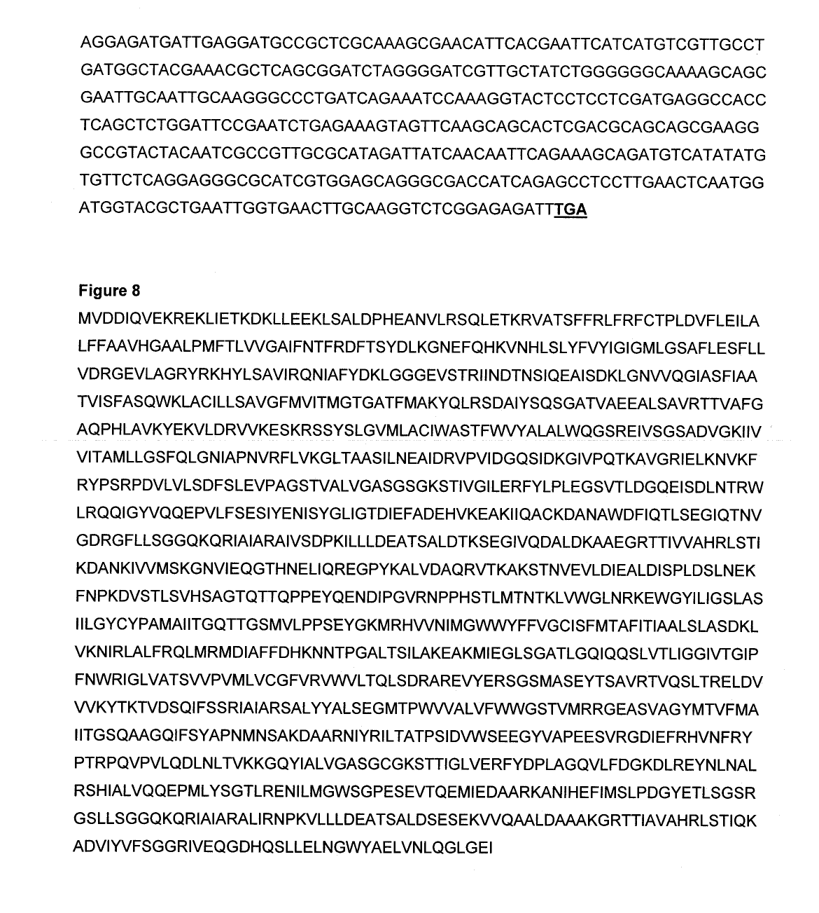

Translation of this large gene results in a protein of 1299 amino acids (FIG. 8, SEQ ID NO:2) and assuming no post-translational modifications, this corresponds with a molecular weight of 142 kDa and a pI of 6.38. The protein shows up to 49% identity with ABC multidrug resistance transporters (MDR) of several Aspergillus species. These transporters take part in the efflux of xenobiotics and/or the secretion of antibiotics. AtrDp from Aspergillus nidulans, for instance, enhances resistance against cytotoxic components and is at the same time required for efficient penicillin secretion (Andrade et al., 2000).

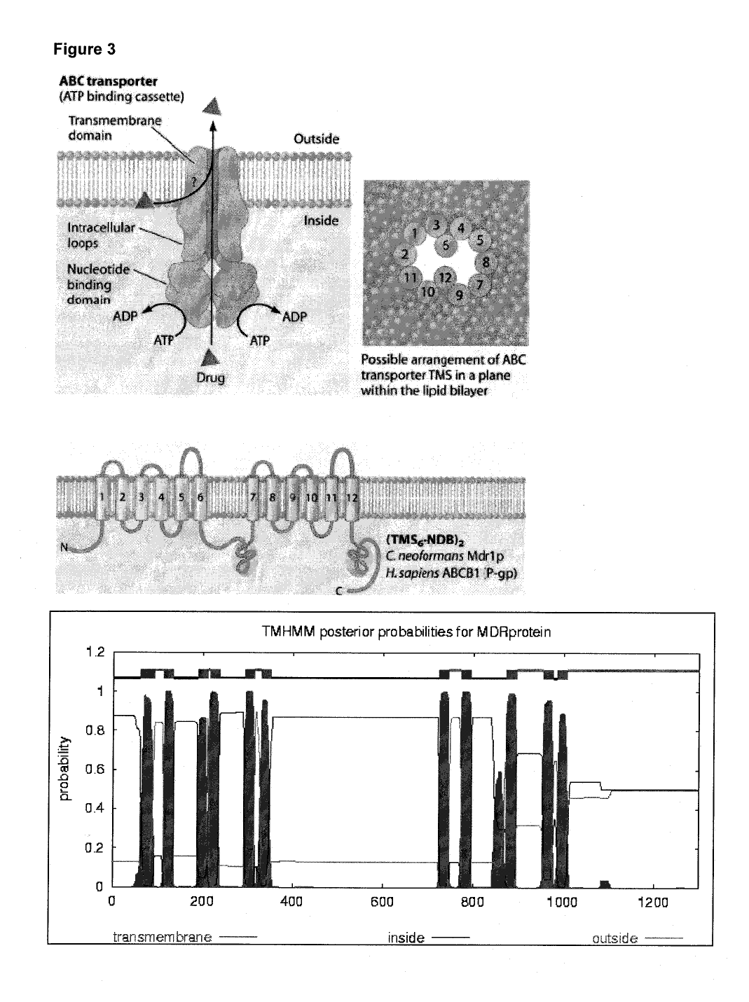

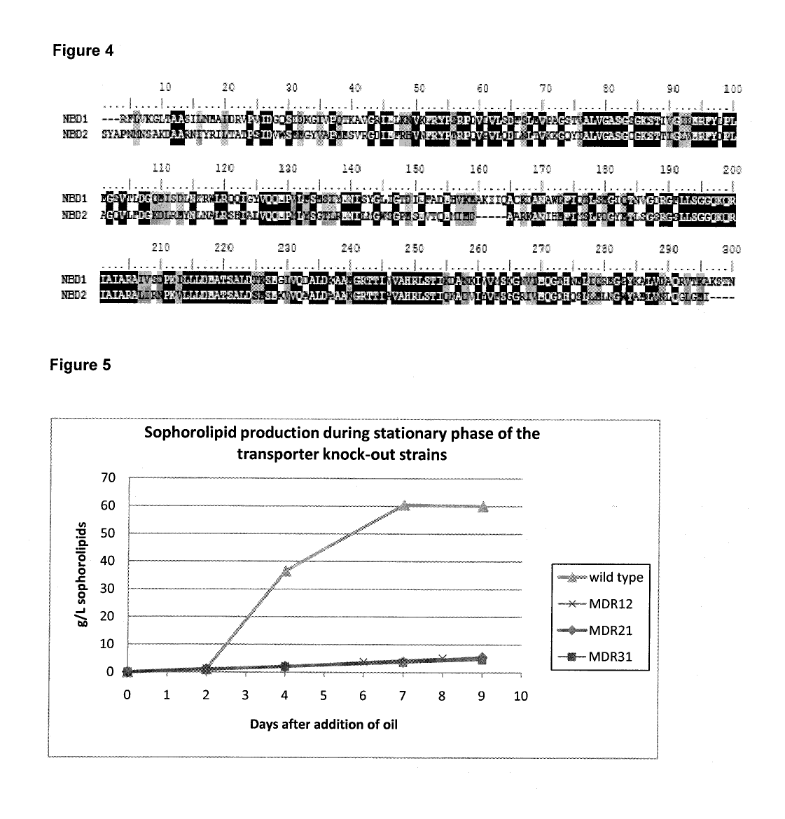

Being transporters, MDR proteins are membrane integrated. Analysis of the amino acid sequence suggested the presence of 12 transmembrane helixes (TM; Kroch et al., 2001) and two nucleotide binding domains (NBD; Zdobnov & Apweiler, 2001) arranged in the characteristic homodimeer-like (TM₆-NBD)₂ MDR structure (FIG. 3). When comparing the two halves of the enzyme, there is a striking similarity between them; it is believed that the transporters emerged from a true homodimeer after gene duplication and fusion. For example, the MDR Sav1866 from Staphylococcus aureus has a TM₆-NBD structure and appears as a homodimeer (Dawson et al., 2006). As presented in FIG. 3, the active part of the transporter is located in the cytosol and, in agreement with this, the intracellular loops, including the NBDs, are highly conserved when compared intra- or intermolecular, whereas the TM regions and extracellular loops show higher diversity. FIG. 4 shows the alignment of the two NBDs of the C. bombicola sophorolipid transporter. The conserved amino acid sequences for ATP binding, the Walker A and B motifs and the ABC signature sequence, are present (Walker et al., 1982).

### Example 2

### Creation and Evaluation of the Knock-Out Strain

The sophorolipid transporter knock-out cassette was constructed as described in the Materials and Methods section. This linear fragment was used to transform the ura3-negative Candida bombicola PT36 strain. The genotype of the transformants was checked by yeast colony PCR with two primer pairs. The first combination, MDRinsertCheckUp and Ura3up.n, verifies the upstream recombination event; MDRinsertCheckUp binds the genomic DNA preceding the integration region and Ura3up.n binds the marker gene of the disruption cassette. The second pair checks the downstream part in the some way: MDRinsertCheckDown binds the genomic region, whereas ura3OutEndRev binds the marker gene. Five out of 31 colonies displayed the desired genotype.

The mutants were first evaluated for their resistance toward several antibiotics. Candida bombicola is known to be highly resistant toward several antibiotics commonly used in yeast research (Van Bogaert, 2008). Until now, only hygromycin can be used as a dominant drug selective marker, while the yeast keeps growing in the presence of high concentrations of G418, zeocin and phleomycin (e.g., >1400 μg/mL G418, whereas 200 μg/mL is sufficient to kill S. cerevisiae). Different cell concentrations of all five mutant strains were put on solid media containing pleomycin, G418 or zeocin. No difference could be observed between the wild-type and the mutants; growth was observed at the same time points and for the same cell concentrations. This finding strengthened the hypothesis that the sophorolipid transporter was not directly involved in the high resistance phenotype, but is assigned a specific role in sophorolipid transport.

If the transporter takes part in sophorolipid export, knocking out the gene should result in reduced sophorolipid production or even toxicity for the producing cell. Sophorolipid synthesis of three genetically identical mutants (MDR12, MDR21 and MDR31) was evaluated on rapeseed oil; the preferred hydrophobic carbon source for high sophorolipid yield. A first indication for reduced sophorolipid production is a decrease in glucose consumption. While in the first part of the stationary phase glucose consumption of the wild-type and the mutants is more or less the same, there is a clear difference in the latter part; glucose is consumed much faster by the wild-type. Indeed, quantification of the sophorolipid synthesis revealed a significant difference between the wild-type and the mutants; although sophorlipids were still detected, they never reached more than 10% of the wild-type titer (FIG. 5).

It must be stressed that cell growth or viability of the mutants was not affected; CFU, CDW and cell shape were similar to the wild-type.

### Example 3

### Creation and Evaluation of the Over-Expression Strain

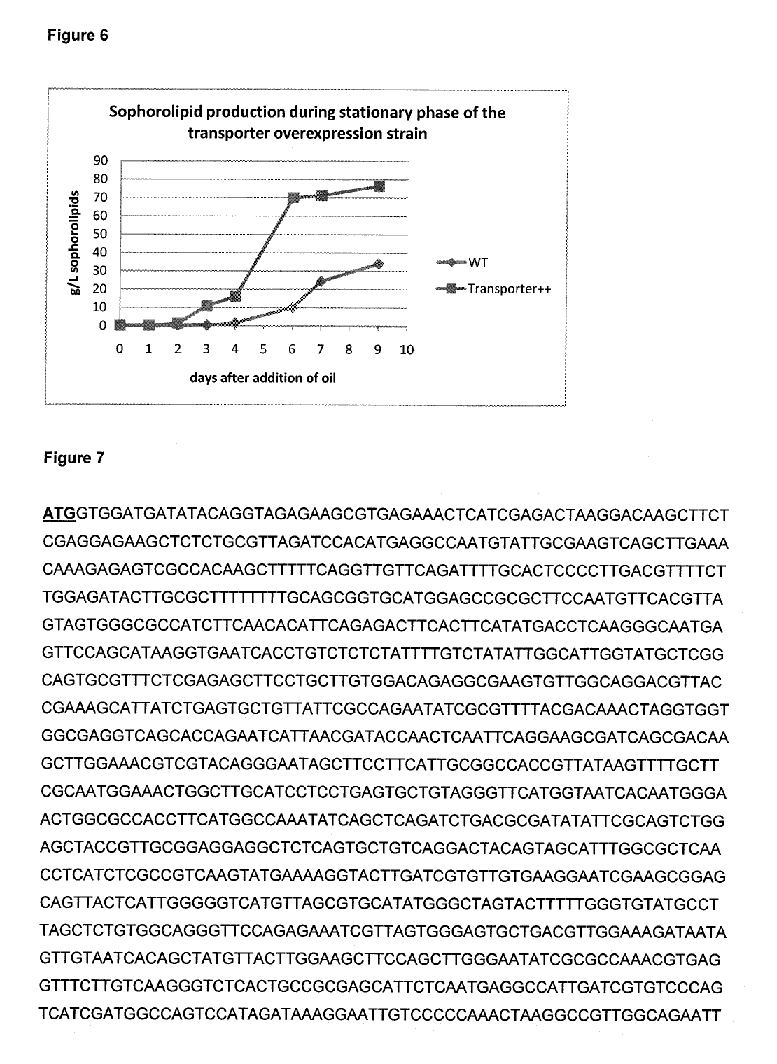

The sophorolipid transporter over-expression cassette was constructed as described in the Materials and Methods section. This linear fragment was used to transform the ura3-negative Candida bombicola PT36 strain. The genotype of the transformants was checked by yeast colony PCR with two primer pairs. The first combination, MDRinsertCheckUp and ura3 5′ REV, verifies the upstream recombination event; MDRinsertCheckUp binds the genomic DNA preceding the integration region and ura3 5′ REV binds the marker gene of the disruption cassette. The second pair checks the downstream part in the same way: MDRCheck2REV binds the genomic region, whereas GAPDhygro194 binds the insert. The production of sophorolipids of a correct transformant strain was compared to the wild-type on medium according to Lang et al. (2001). The strain over-expressing the transporter showed an increased secretion of sophorolipids when compared with the non-transformed parental strain, cultivated under the same conditions (FIG. 6). Biomass formation measure by CDW and cell viability determined by CFU were similar to the parental strain, demonstrating that the increased yields were not caused by increased biomass and that augmented production had no negative effect on cell viability.

### Example 4

### Use of the Sophorolipid Transporter to Increase Cellobiose Lipid Synthesis in Ustilago maydis

U. maydis DSM17146 was transformed with either the p1025 expression plasmid harboring the transporter, a digest hereof or a PCR fragment derived hereof as described in the material and methods section. For each of the three transformations, four colonies appearing on the selective plates were grown in non-selective medium (YPD) to screen for a stable integration event and gDNA was isolated. The presence of the construct was verified by PCR with the primers GPDumFor and MDRinsertCheckREV and all four plasmid-derived transformants harbored the construct as well as all PCR-derived ones. Two out of four digest-derived ones were positive as well.

These two latter strains, as well as three randomly selected strains from the plasmid-derived ones, and three randomly selected strains from PCR-derived ones, were tested for their cellobiose lipid production as described in the material and methods section.

### REFERENCES

Altschul S. F., T. L. Madden, et al. (1997). “Gapped BLAST and PSI-BLAST: a new generation of protein database search programs.” Nucleic Acids Research 25:3389-3402.
Altschul S. F., J. C. Wootton, et al. (2005). “Protein database searches using compositionally adjusted substitution matrices.” FEBS Journal 272:5101-5109.
Andrade A C; J. G. M. Van Nistelrooy, et al. (2000). “The role of ABC transporters from Aspergillus nidulans in protection against cytotoxic agents and in antibiotic production.” Molecular and General Genetics 263:966-977.
Brachmann A., J. König, et al. (2004). “A reverse genetic approach for generating gene replacement mutants in Ustilago maydis.” Molecular and General Genomics 272:216-226.
Cannon R. D., E. Lamping, et al. (2009). “Efflux-Mediated Antifungal Drug Resistance.” Clinical Microbiology Reviews 22:291-321.
Dawson R. J. and K. P. Locher (2007). “Structure of the multidrug ABC transporter Sav1866 from taphylococcus aureus in complex with AMPPNP.” FEBS Letters 581:935-938.
De Maeseneire S. L., I. N. Van Bogaert, et al. (2007). “Rapid isolation of fungal genomic DNA suitable for long distance PCR.” Biotechnology Letters 29:1845-1855.
Gietz R. D. and R. H. Schiestl (1995). “Transforming yeast with DNA.” Methods In Molecular And Cellular Biology 5:255-269.
Inoue H., H. Nojima, and H. Okayama (1990). “High efficiency transformation of Escherichia coli with plasmids.” Gene 96:23-28.
Krogh A., B. Larsson, et al. (2001). “Predicting transmembrane protein topology with a hidden Markov model: Application to complete genomes.” Journal of Molecular Biology 305:567-580.
Lang S., A. Brakemeier, et al. (2000). “Production of native and modified sophorose lipids.” Chimica Oggi-Chemistry Today 18:76-79.
Morgulis A., Coulouris, et al. (2008). “Database Indexing for Production MegaBLAST Searches.” Bioinformatics 24:1757-1764.
Spoeckner S., V. Wray, et al. (1999). “Glycolipids of the smut fungus Ustilago maydis from cultivation on renewable resources.” Applied Microbiology and Biotechnology 51:33-39.
Teichmann B., U. Linne, et al. (2007). “A biosynthetic gen cluster for a secreted cellobiose lipid with antifungal activity from Ustilago maydis.” Molecular Microbiology 66:525-533.
Tobin M. B., R. B. Peery, et al. (1997). “Genes encoding multiple drug resistance-like proteins in Aspergillus fumigatus and Aspergillus flavus.” Gene 200:11-23.
Van Bogaert I. N. A., K. Saerens, et al. (2007). “Microbial production and application of sophorolipids.” Applied Microbiology and Biotechnology 76:23-34.
Van Bogaert I. N. A., S. L. De Maeseneire, et al. (2008). “Development of a transformation and selection system for the glycolipid-producing yeast Candida bombicola.” Yeast 25:273-278.
Van Bogaert I. N. A. (2008). “Microbial synthesis of sophorolipids by the yeast Candida bombicola.” PhD-thesis, Faculty of Bioscience Engineering, Ghent University, Ghent, Belgium, 239 p.
Van Bogaert I. N. A., M. Demey, et al. (2009a). “Importance of the cytochrome P450 monooxygenase CYP52 family for the sophorolipid-producing yeast Candida bombicola.” FEMS Yeast Research 9:87-94.
Van Bogaert I. N. A., J. Sabirova, et al. (2009b). “Knocking out the MFE-2 gene of Candida bombicola leads to improved medium-chain sophorolipid production.” FEMS Yeast Research 9:610-617.
Walker J. E., M. Saraste, et al. (1982). “Distantly Related Sequences in the Alpha-Subunits and Beta-Subunits of Atp Synthase, Myosin, Kinases and Other ATP-Requiring Enzymes and a Common Nucleotide Binding Fold.” EMBO Journal 1:945-951.
Zdobnov E. M. and R. Apweiler (2001). “InterProScan—an integration platform for the signature-recognition methods in InterPro.” Bioinformatics 17:847-848.
Zhang Z., S. Schwartz, et al. (2000). “A greedy algorithm for aligning DNA sequences.” Journal of Computational Biology 7:203-14.

What is claimed is:

1. (canceled)

2. An isolated sophorolipid transporter protein having at least 70% identity to SEQ ID NO:2.

3. The sophorolipid transporter protein of claim 2, which is isolated from Candida bombicola.

4. An isolated nucleic acid molecule encoding the sophorolipid transporter protein of claim 1, or a functional fragment thereof.

5. The nucleic acid molecule of claim 4, having at least 70% identity to SEQ ID NO:1, or a functional fragment thereof.

6. A host organism, transformed with the nucleic acid molecule of claim 4.

7. The host organism according to claim 6, wherein said host organism is a fungal strain.

8. A method of modulating glycolipid secretion and/or production in a host, the method comprising:
utilizing an isolated sophorolipid transporter protein according to claim 1 so as to modulate glycolipid secretion and/or production in the host.

9. The method according to claim 8, wherein said modulation is an increase in secretion and/or production of glycolipids.

10. A method of modulating glycolipid secretion and/or production in a host, the method comprising:
utilizing the nucleic acid according to claim 4 so as to modulate the glycolipid secretion and/or production in the host.

11. The method according to claim 10, wherein said modulation is an increase in secretion and/or of glycolipids.

12. The method according to claim 8, wherein said glycolipid is a sophorolipid or a cellobiose lipid, or a biochemical modification of sophorolipid or cellobiose lipid.

13. A method for increasing secretion and/or production of glycolipids in a host organism, the method comprising:
transforming the host organism with the nucleic acid according to claim 4.

14. The method according to claim 13, wherein said host organism is a fungus.

15. The method according to claim 14, wherein said fungus is Candida bombicola.

16. The method according to claim 15, wherein said glycolipid is a sophorolipid.

17. The method according to claim 14, wherein said fungus is Ustilago maydis.

18. The method according to claim 17, wherein said glycolipid is a cellobiose lipid.

19. A method for increasing secretion and/or production of sophorolipid and/or a cellobiose lipid in a fungus, the method comprising:
transforming the fungus with a nucleic acid molecule having at least 70% identity to SEQ ID NO:1
so as to increase secretion and/or production of sophorolipid and/or cellobiose lipid in the fungus.

20. The method according to claim 14, wherein the fungus is Candida bombicola.

21. The method according to claim 14, wherein the fungus is Ustilago maydis.
